(19) United States
(12) Patent Application Publication
(10) Pub. No.: US 2021/0055942 A1
(43) Pub. Date: Feb. 25, 2021

(54) SERVER AND SYSTEM FOR MULTI-LANGUAGE SUPPORT FOR SOFTWARE IN THE CONTEXT OF MULTI-TENANCY

(71) Applicant: nPhase, Inc., Encinitas, CA (US)

(72) Inventor: Jaya Prakash R. Guda, Cupertino, CA (US)

(21) Appl. No.: 17/000,025

(22) Filed: Aug. 21, 2020

Related U.S. Application Data
(60) Provisional application No. 62/889,665, filed on Aug. 21, 2019.

(51) Int. Cl. G06F 9/451; G16H 10/20; G06F 40/58; G06F 3/0484

(52) U.S. Cl. CPC G06F 9/454; G06F 3/04842; G06F 40/58; G16H 10/20

(57) ABSTRACT

This disclosure describes a system for performing multinational clinical trials and data collection for each trial. In some embodiments, the data collected will be collected in the current language for each specific user. In some embodiments, data collected from each country can be collected in separate categories or in different ways. In some embodiments, a form is presented in a language that is dependent on the geolocation of a computer accessing the form. For example, forms accessed in the United States will appear in English, data collected in Mexico will appear in Spanish, etc. In some embodiments, a user can enable the data collection form to be presented in multiple languages. In some embodiments, the user can translate the form and input text into different languages, and into multiple languages simultaneously.

### CROSS REFERENCE

This application claims the benefit of U.S. Provisional Patent Application No. 62/889,665, filed Aug. 21, 2019, the entire contents of which are incorporated by reference herein.

### BACKGROUND

Some embodiments of the disclosed technologies relate generally to the field of computer technologies. More particularly, some embodiments relate generally to maintaining multiple versions of Software as a Service (SaaS) applications (e.g., cloud-computing based software) in the context of multi-tenancy and application management systems.

Globalization is increasingly shaping the life science and virtually all other industries. To study medical conditions and treatments, Clinical Research Organizations (CROs) and pharmaceutical companies need to carry out scientific trials all around the world to get an accurate and representative population for testing. This usually involves groups of people of different ages, genders and cultures.

To conduct these trials, they need to take a multilingual approach: Informed Consent Forms (ICFs), Patient Information Sheets (PIS), investigator's brochures, study protocols and amendments, agreements and authorizations, questionnaires and patient diaries need to be available in several languages. Translation is now regarded as a fundamental step throughout clinical trial processes, especially for international research projects.

The current approach for handling language issues using eClinical Systems for Global Clinical Trials is to develop different data collection forms for each language. Some implications are: Changes in the data collection form (adding/deleting new data fields, label changes, validation logic, etc.) will necessitate changes to the all the forms in all languages. Each of these language forms will have to be added to different data collection events, creating study complexity. Rules and triggers on the data collection events need to be duplicated, and any changes to the rules and triggers will have to applied across all the forms in all languages. Further, reporting is very difficult, as the data collection is in different underlying database structures.

### SUMMARY

Some embodiments enable users to handle language issues in Global Clinical Trials by developing a single data collection form and having the ability to support the form in multiple languages. In some embodiments, changes in the data collection form (adding/deleting new data fields, label changes, validation logic) require changes in only one form, simplifying the overall study design. In some embodiments, study complexity is reduced as only one form is added to the Data Collection Event, and there are no missed forms. In some embodiments, rules and triggers on the data collection events do not have to be duplicated. Further, some embodiments include changes to any rule or trigger that is applied across all forms in all languages. In some embodiments reporting is simplified, as the data collection single underlying database structure.

Some embodiments of the invention reduce costs for Clinical Trials. Some embodiments include: streamlined study design, reduced Q&A and user acceptance testing, reduced user training, and reduced on-going maintenance and operations of the study.

Some embodiments include a form designer tool which is compatible with and can use multiple languages. Some embodiments include a form preview. Further, some embodiments include data collection and reporting.

In some embodiments, the system is configured to collect data from multinational clinical trials. In some embodiments, the system comprises one or more computers comprising one or more processors and one or more computer readable media. In some embodiments, the one or more computer readable media comprise processor executable instructions that when executed by the one or more processors implement a graphical user interface (GUI) and/or a translation module. In some embodiments, the graphical user interface (GUI) configured to display a form. In some embodiments, the form is configured to display and/or collect clinical trial information. In some embodiments, the clinical trial information comprises an input language and a translated language. In some embodiments, the system is configured to receive the input language from the form and translate the input language to the translated language.

In some embodiments, the one or more computer readable media further comprise processor executable instructions that when executed by the one or more processors implement one or more of a geolocation module, a comparison module, and a language list. In some embodiments, the language list comprises a list of one or more languages used in one or more geographical locations. In some embodiments, the geolocation module is configured to receive a geolocation of one or more remote computers displaying the form. In some embodiments, the comparison module is configured to compare the geolocation to the one or more geographical locations. In some embodiments, if the geolocation matches the one or more geographical locations, the system is configured to display at least a portion of the form in at least one of the one or more languages.

In some embodiments, the at least a portion of the form is a data collection form. In some embodiments, the data collection form is configured to display text associated with the data collection form in at least one of the one or more languages if the geolocation matches the one or more geographical locations. In some embodiments, the system is configured to enable a user to translate the input language to the translated language. In some embodiments, the system is configured to enable a user to translate the input language to the translated language. In some embodiments, at least one of the multiple languages is the translated language.

In some embodiments, the data collection form is configured to display text associated with at least a portion of the form in the input language in response to the input language being detected and/or identified. In some embodiments, at least a portion of the form includes a data collection form, a selection icon, a menu, instructions, alerts, and/or any component of the form described and/or shown herein.

### BRIEF DESCRIPTION OF THE DRAWINGS

Other systems, methods, features, and advantages will be or will become apparent to one with skill in the art upon examination of the following figures and detailed description. It is intended that all such additional systems, methods, features, and advantages be included within this description, be within the scope, and be protected by the accompanying claims. Component parts shown in the drawings are not necessarily to scale and may be exaggerated to better illustrate the important features. In the drawings, like reference numerals designate like parts throughout the different views, wherein:

FIG. 1 illustrates a screen summary of the user's screen with the ability to enable the data collection form in multiple languages according to some embodiments.

FIG. 2 illustrates a screen summary of the user's screen with an option to add translations in multiple languages according to some embodiments.

FIG. 3 illustrates a screen summary of the user's screen with an option to add translations in multiple languages according to some embodiments.

FIG. 4 illustrates a screen summary of the user's screen showing the translation feature to add the translation in multiple languages according to some embodiments.

FIG. 5 illustrates a screen summary of the user's screen showing the translation in multiple languages according to some embodiments.

FIG. 6 illustrates a screen summary of the user's screen with an option to provide translations for Response Sets according to some embodiments.

FIG. 7 illustrates a screen summary of the user's screen showing the translation feature to add translations for Response Sets in multiple languages according to some embodiments.

FIG. 8 illustrates a screen summary of the user's screen showing the translation of Response Sets in multiple languages according to some embodiments.

FIG. 9 illustrates a screen summary of the user's screen with an option to preview the data collection form in multiple languages according to some embodiments.

FIGS. 10-11 illustrate a screen summary of the translation according to some embodiments.

FIG. 12 illustrates a screen summary of the user's screen showing the ability to collect data collection in multiple languages according to some embodiments.

FIG. 13 illustrates a screen summary of the United States Customs and Border Protection—Form CBP 509B in multiple languages according to some embodiments.

FIG. 14 illustrates a computer server system configured for operating and processing components of the system in accordance with some embodiments.

### DETAILED DESCRIPTION

The detailed description of exemplary embodiments herein makes reference to the accompanying drawings and pictures, which show the exemplary embodiments by way of illustration and its best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

In some embodiments, users perform multinational clinical trials and collect data for each trial. In some embodiments the data collected will be collected in the current language for each specific user. Often, the data collected is in the English language. In some embodiments, users who perform the clinical trials can perform these clinical trials in different countries. In some embodiments, data collected from each country can be collected in separate categories or in different ways. For example, data collected in the United States will appear in English, data collected in Mexico will appear in Spanish.

In some embodiments, users can translate their collected data into their preferred language for each trial. In some embodiments a user who collects data in Spanish can translate their data into the local language of their colleagues who reside outside of the local language where the data have been collected. This provides the users a more streamlined reporting of each clinical trial.

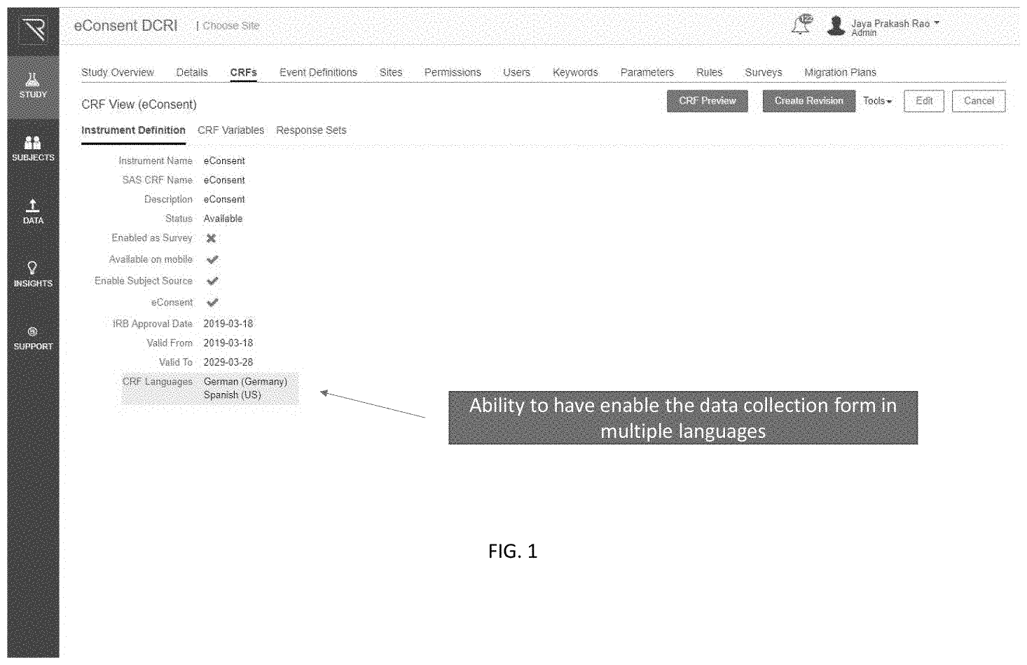

FIG. 1 illustrates a screen summary of the user's screen with the ability to enable the data collection form in multiple languages according to some embodiments. In some embodiments a user can enable multiple languages on the Instrument Definition screen. In some embodiments a user can enable the data collection form to be presented in multiple languages. In some embodiments the user can enable one or more languages for translation at one time.

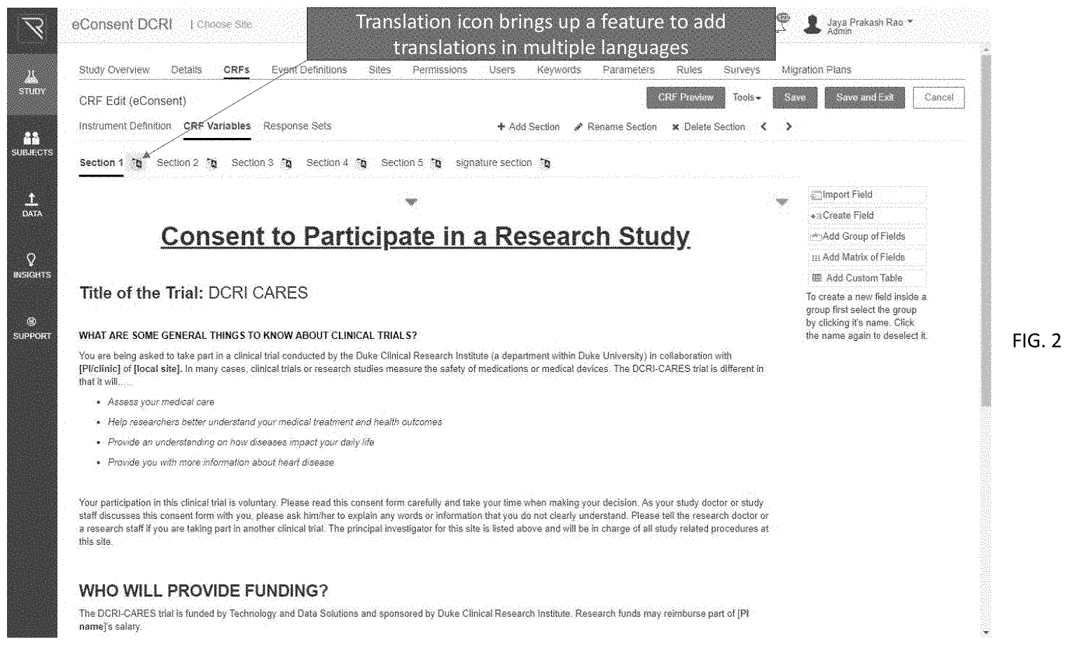

FIG. 2 illustrates a screen summary of the user's screen with an option to add translations in multiple languages according to some embodiments. In some embodiments a user can select a translation icon and/or cube when they are viewing the CFR Variables screen. In some embodiments the translation icon and/or cube enables a feature to add translations in multiple languages. In some embodiments a user can select the translation icon and/or cube in several sections; Section 1, Section 2, Section 3, Section 4, Section 5, and/or the Signature Section.

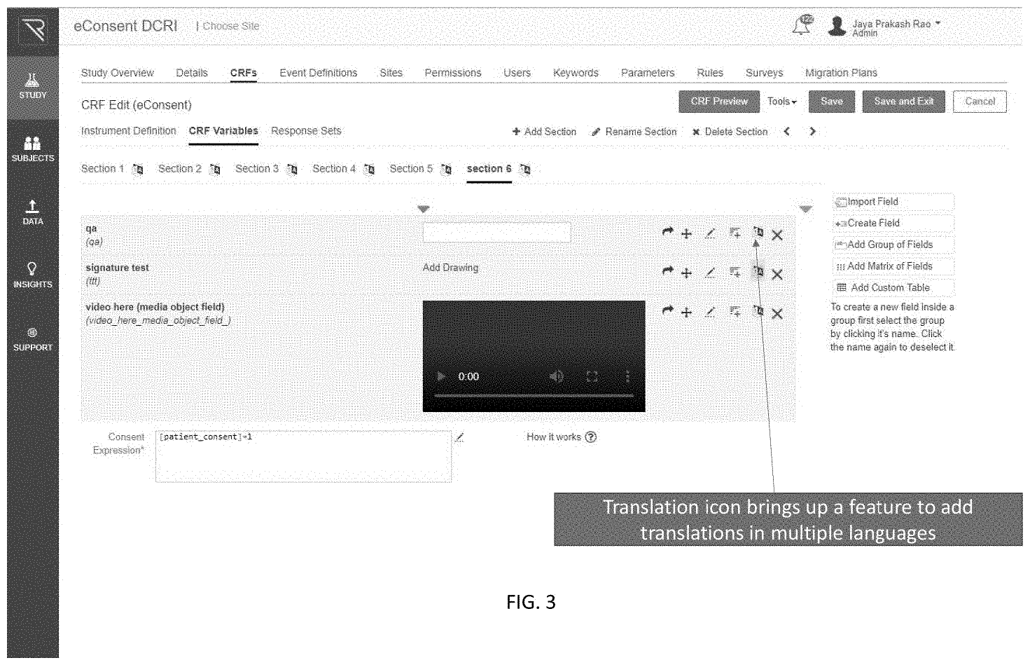

FIG. 3 illustrates a screen summary of the user's screen with an option to add translations in multiple languages according to some embodiments. In some embodiments a user can add data and/or media in a Section on the CFR Variables screen. In some embodiments a user can select the translation icon and/or cube for changing the data and/or media into multiple languages.

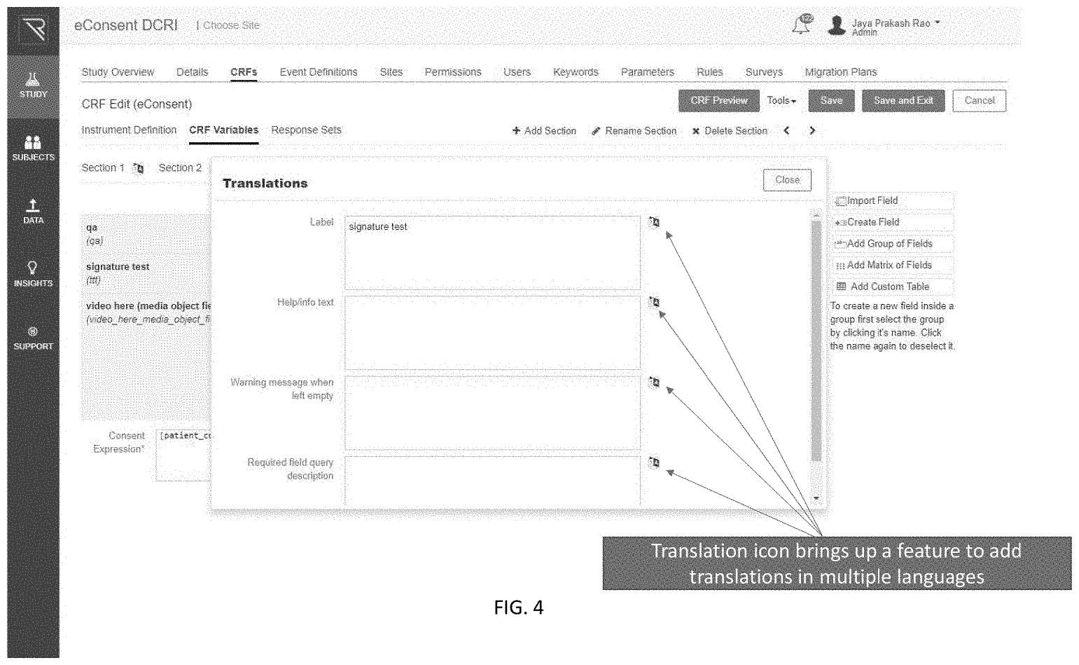

FIG. 4 illustrates a screen summary of the user's screen showing the translation feature to add the translation in multiple languages according to some embodiments. In some embodiments, once the translation icon and/or cube is selected, a translation screen will appear. In some embodiments the translation screen can have multiple areas for data input, for example: a label box, a help/info text box; a warning message when left empty box; and a required field query description box. In some embodiments the translation icon and/or cube can bring up a feature to add translations in multiple languages for one or more of the data input boxes. In some embodiments the user can add data into one or more of the label box; help/info text box; warning message when left empty box; and the required field query description box. Once the data has been added to one or more of the listed boxes, the user can select the translation icon and/or cube for translation in multiple languages.

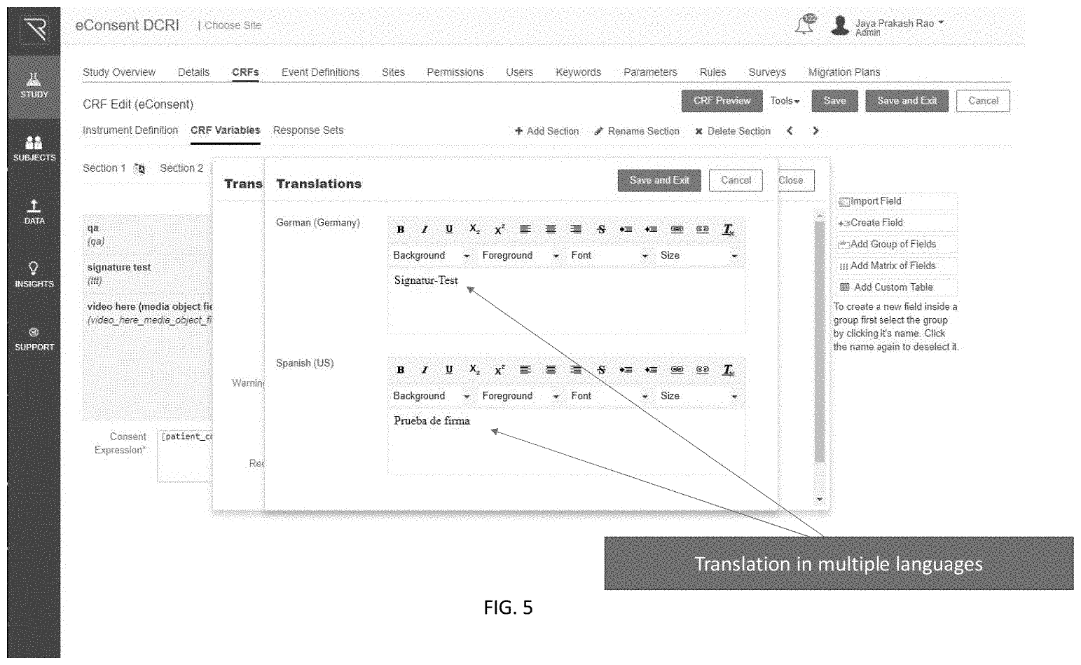

FIG. 5 illustrates a screen summary of the user's screen showing the translation in multiple languages according to some embodiments. In some embodiments, after the translation icon and/or cube has been selected, the user can choose the preferred languages for translation. In some embodiments, the data can appear in one or more languages.

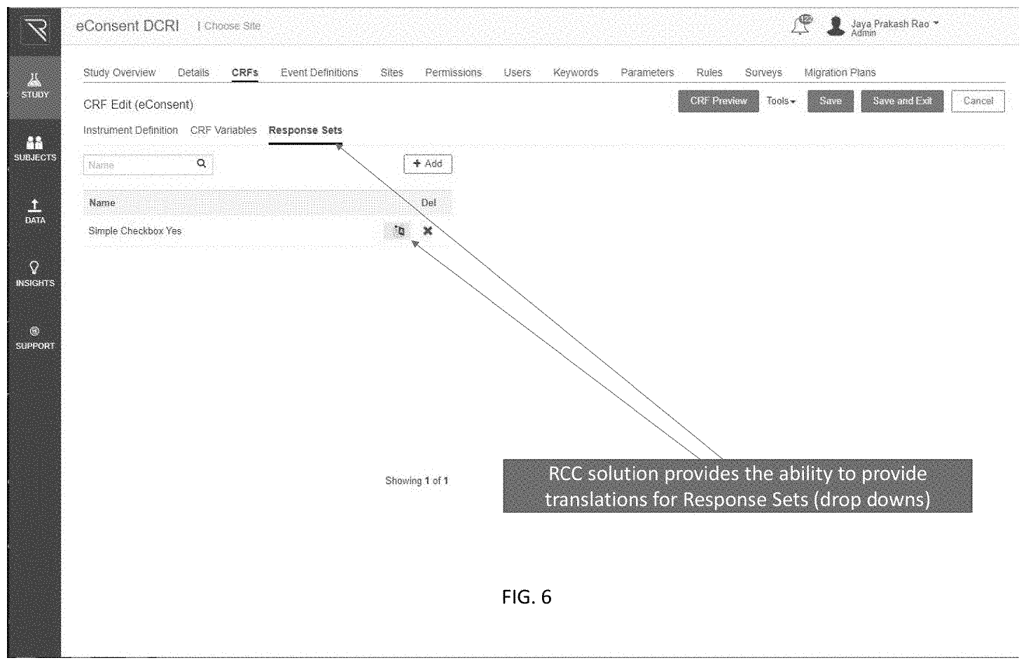

FIG. 6 illustrates a screen summary of the user's screen with an option to provide translations for Response Sets according to some embodiments. In some embodiments when a user is on the Response Sets screen, the user can select the specific response set for translation. In some embodiments the translation icon and/or cube can be selected for one or more response sets.

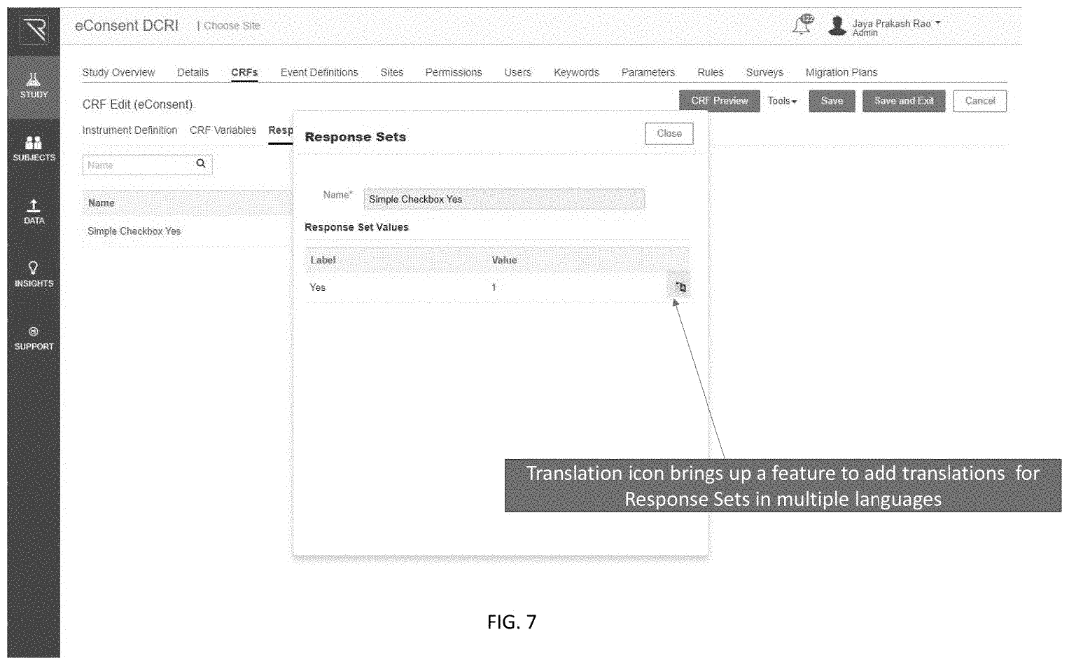

FIG. 7 illustrates a screen summary of the user's screen showing the translation feature to add translations for Response Sets in multiple languages according to some embodiments. In some embodiments a user has selected the translation icon and/or cube for one or more response sets. In some embodiments once the response set is selected, the translation icon brings up a feature to add translations in multiple languages. In some embodiments the translation icon and/or cube can be selected for each specific response set.

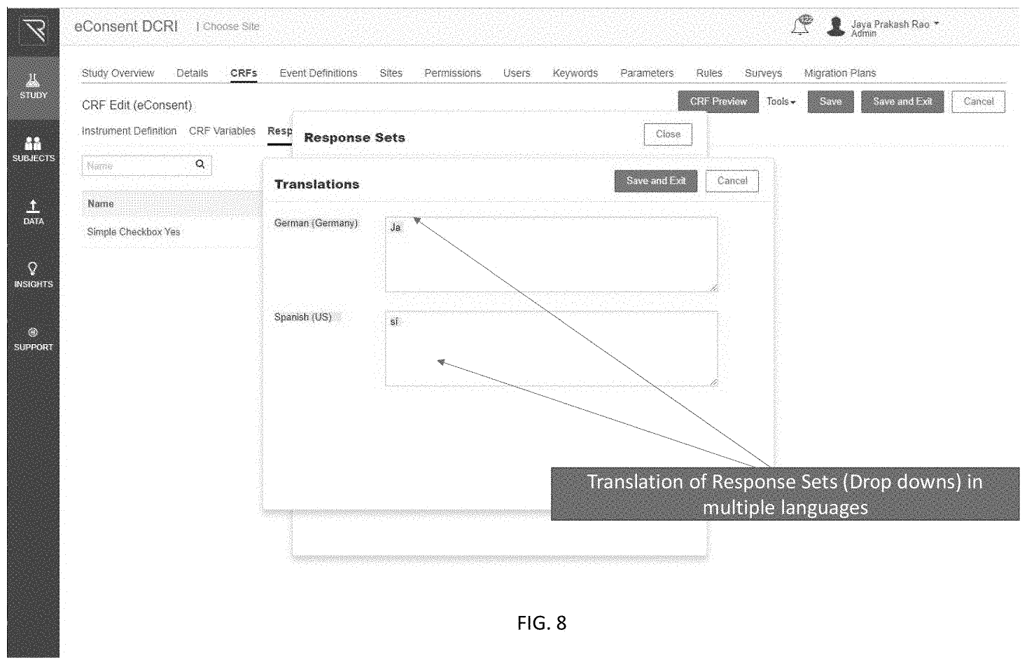

FIG. 8 illustrates a screen summary of the user's screen showing the translation of Response Sets in multiple languages according to some embodiments. In some embodiments once the translation icon and/or cube is selected in one or more response sets, the translation screen will appear. In some embodiments the translation screen will provide the user with the translation in the languages selected.

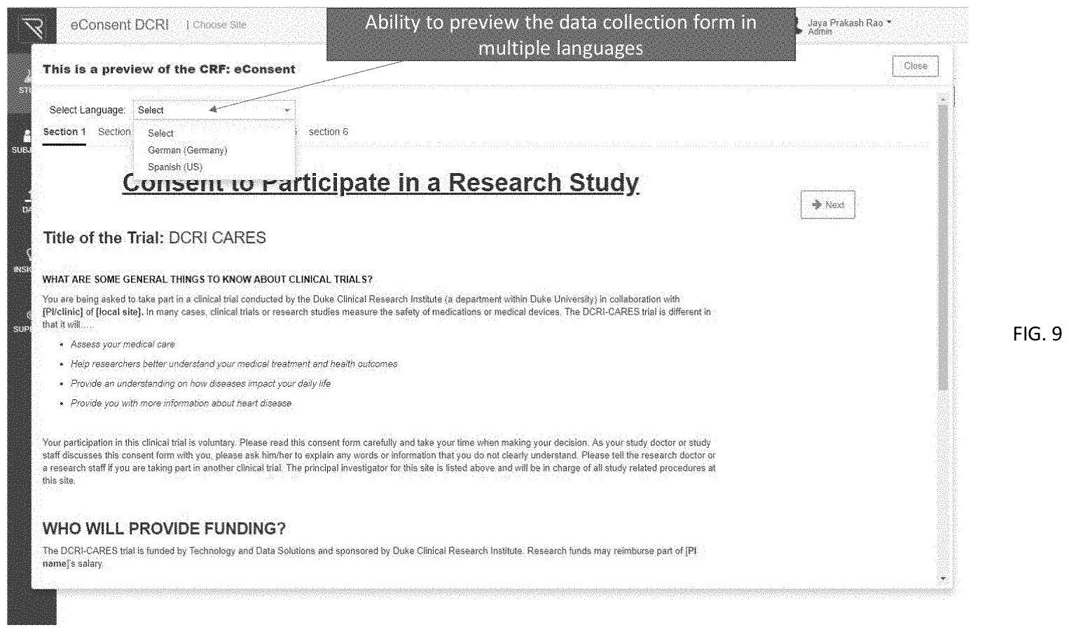

FIG. 9 illustrates a screen summary of the user's screen with an option to preview the data collection form in multiple languages according to some embodiments. In some embodiments a user can select a specific language to preview the data collected.

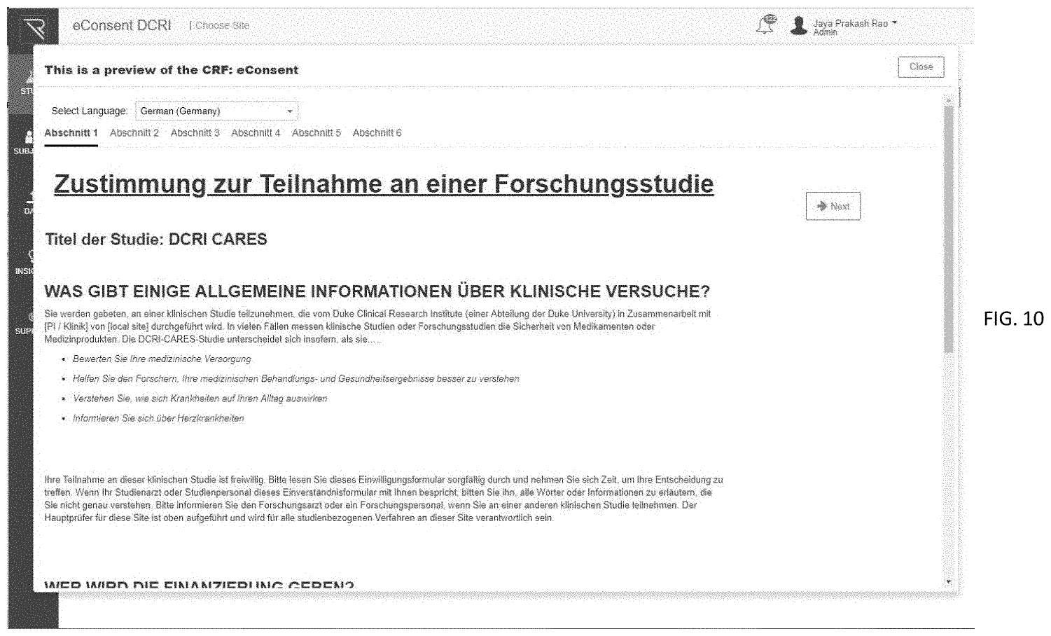

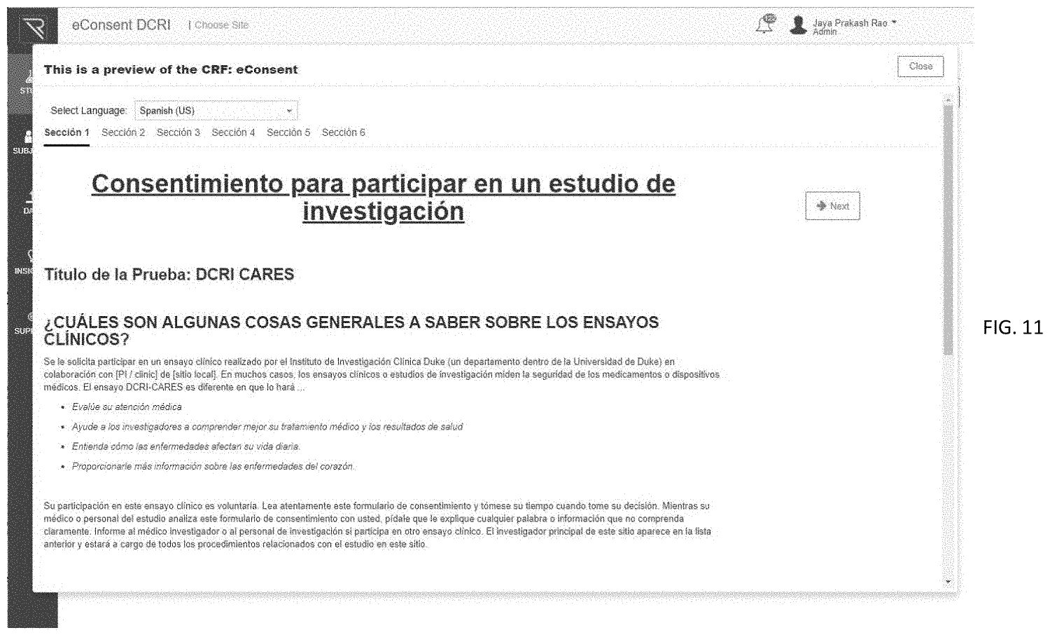

FIGS. 10-11 illustrate a screen summary of the translation according to some embodiments. In some embodiments, the user can select a specific language to preview the data. In some embodiments once the user has selected a specific language, the data collected will previewed in that specific language. For example, if the user has selected the translation to be in German, the user's data will be translated into German.

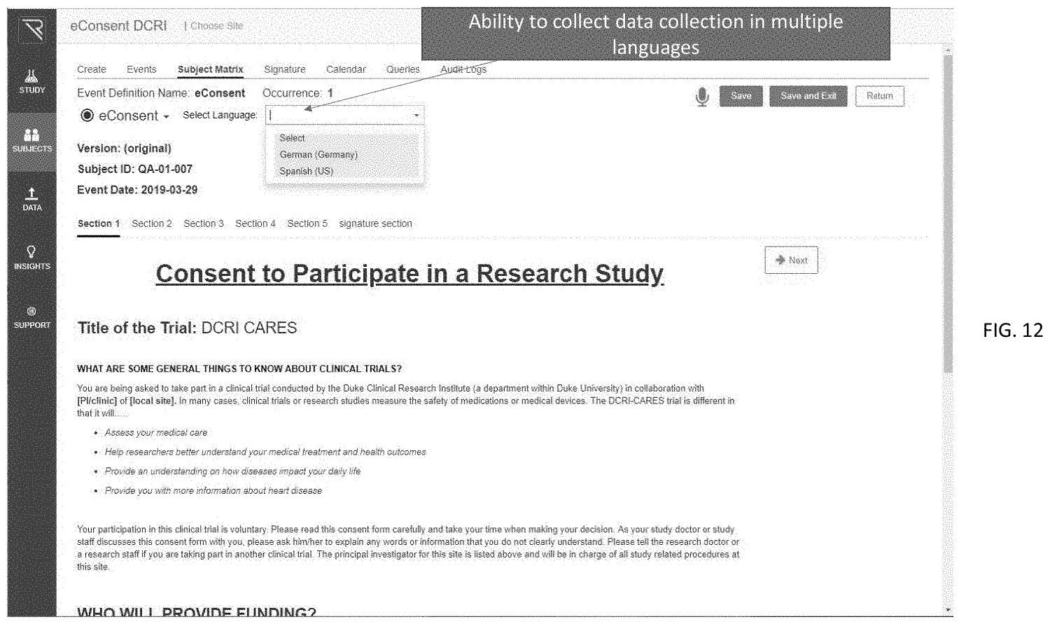

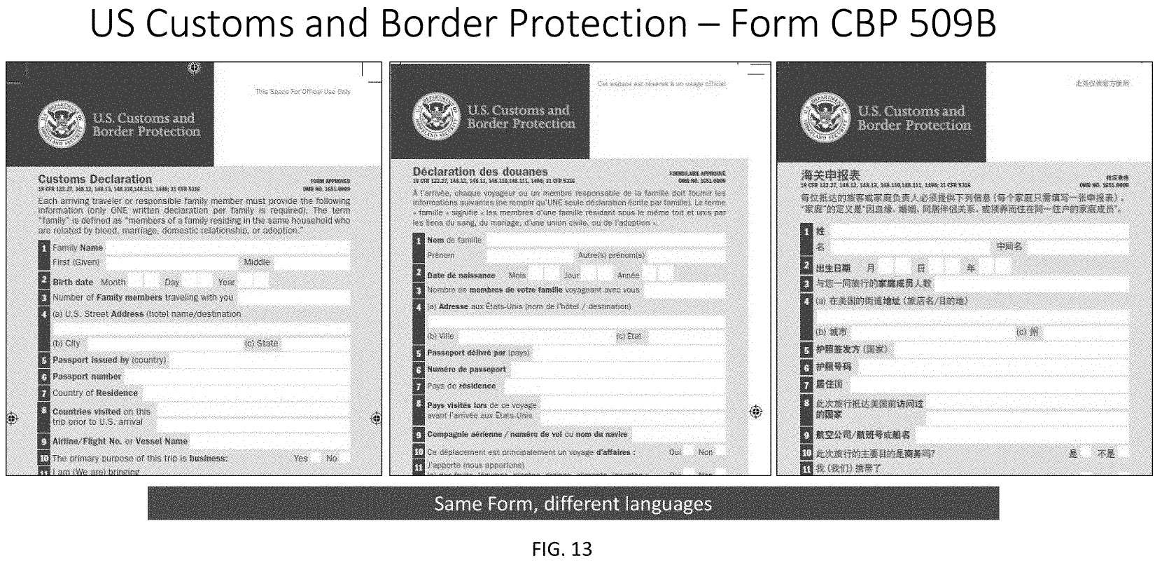

FIG. 12 illustrate a screen summary of the user's screen showing the ability to collect data in multiple languages according to some embodiments. In some embodiments the user can collect data in multiple languages on the eConsent form.

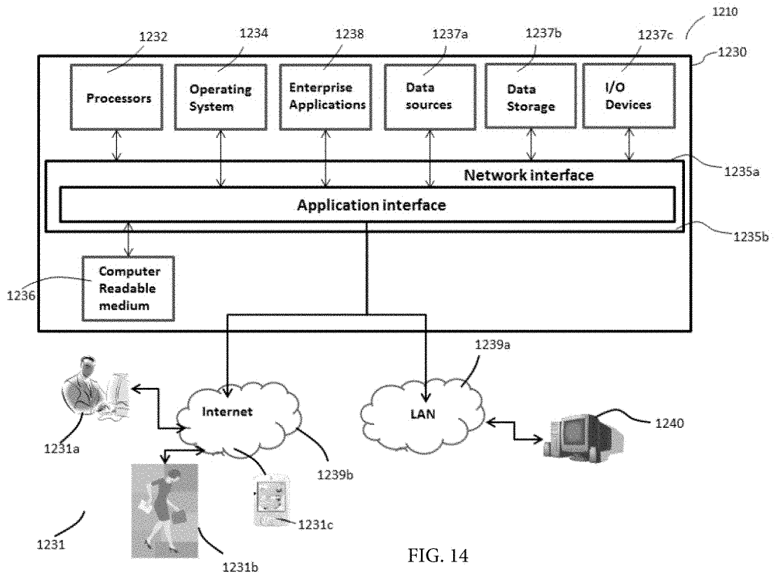

FIG. 14 illustrates a computer system 1210 configured for operating and processing components of any of the systems or portions of the systems described earlier and illustrated in FIGS. 1-12. In some embodiments, the computer system 1210 can operate and/or process computer-executable code of one or more software modules of the system described earlier. Further, in some embodiments, the computer system 1210 can operate and/or display information related to one or more graphical user interfaces. In some embodiments, the system 1210 can comprise at least one computing device including at least one processor 1232. In some embodiments, the at least one processor 1232 can include a processor residing in, or coupled to, one or more server platforms. In some embodiments, the system 1210 can include a network interface 1250a and an application interface 1250b coupled to the least one processor 1232 capable of processing at least one operating system 1240. Further, in some embodiments, the interfaces 1250a, 1250b coupled to at least one processor 1232 can be configured to process one or more of the software modules (e.g., such as enterprise applications 1238). In some embodiments, the software modules 1238 can include server-based software modules. In some embodiments, the software modules 1238 can operate to host at least one user account and/or at least one client account, and operating to transfer data between one or more of these accounts using the at least one processor 1232.

With the above embodiments in mind, it should be understood that the invention can employ various computer-implemented operations involving data stored in computer systems. Moreover, the above-described databases and models throughout the system can store analytical models and other data on computer-readable storage media within the system 1210 and on computer-readable storage media coupled to the system 1210. In addition, the above-described applications of the system can be stored on computer-readable storage media within the system 1210 and on computer-readable storage media coupled to the system 1210. These operations are those requiring physical manipulation of physical quantities. Usually, though not necessarily, these quantities take the form of electrical, electromagnetic, or magnetic signals, optical or magneto-optical form capable of being stored, transferred, combined, compared and otherwise manipulated. In some embodiments of the invention, the system 1210 can comprise at least one computer readable medium 1236 coupled to at least one data source 1237a, and/or at least one data storage device 1237b, and/or at least one input/output device 1237c. In some embodiments, the invention can be embodied as computer readable code on a computer readable medium 1236. In some embodiments, the computer readable medium 1236 can be any data storage device that can store data, which can thereafter be read by a computer system (such as the system 1210). In some embodiments, the computer readable medium 1236 can be any physical or material medium that can be used to tangibly store the desired information or data or instructions and which can be accessed by a computer or processor 1232. In some embodiments, the computer readable medium 1236 can include hard drives, network attached storage (NAS), read-only memory, random-access memory, FLASH based memory, CD-ROMs, CD-Rs, CD-RWs, DVDs, magnetic tapes, other optical and non-optical data storage devices. In some embodiments, various other forms of computer-readable media 1236 can transmit or carry instructions to a computer 1240 and/or at least one user 1231, including a router, private or public network, or other transmission device or channel, both wired and wireless. In some embodiments, the software modules 1238 can be configured to send and receive data from a database (e.g., from a computer readable medium 1236 including data sources 1237a and data storage 1237b that can comprise a database), and data can be received by the software modules 1238 from at least one other source. In some embodiments, at least one of the software modules 1238 can be configured within the system to output data to at least one user 1231 via at least one graphical user interface rendered on at least one digital display.

In some embodiments of the invention, the computer readable medium 1236 can be distributed over a conventional computer network via the network interface 1250a where the system embodied by the computer readable code can be stored and executed in a distributed fashion. For example, in some embodiments, one or more components of the system 1210 can be coupled to send and/or receive data through a local area network (“LAN”) 1239a and/or an internet coupled network 1239b (e.g., such as a wireless internet). In some further embodiments, the networks 1239a, 1239b can include wide area networks (“WAN”), direct connections (e.g., through a universal serial bus port), or other forms of computer-readable media 1236, or any combination thereof.

In some embodiments, components of the networks 1239a, 1239b can include any number of user devices such as personal computers including for example desktop computers, and/or laptop computers, or any fixed, generally non-mobile internet appliances coupled through the LAN 1239a. For example, some embodiments include personal computers 1240a coupled through the LAN 1239a that can be configured for any type of user including an administrator. Some embodiments can include personal computers coupled through network 1239b. In some further embodiments, one or more components of the system 1210 can be coupled to send or receive data through an internet network (e.g., such as network 1239b). For example, some embodiments include at least one user 1231 coupled wirelessly and accessing one or more software modules of the system including at least one enterprise application 1238 via an input and output (“I/O”) device 1237c. In some embodiments, the system 1210 can enable at least one user 1231 to be coupled to access enterprise applications 1238 via an I/O device 1237c through LAN 1239a. In some embodiments, the user 1231 can comprise a user 1231a coupled to the system 1210 using a desktop computer, and/or laptop computers, or any fixed, generally non-mobile internet appliances coupled through the internet 1239b. In some embodiments, the user 1231 can comprise a mobile user 1231b coupled to the system 1210. In some embodiments, the user 1231b can use any mobile computing device 1231c to wireless coupled to the system 1210, including, but not limited to, personal digital assistants, and/or cellular phones, mobile phones, or smart phones, and/or pagers, and/or digital tablets, and/or fixed or mobile internet appliances.

In some embodiments of the invention, the system 1210 can enable one or more users 1231 coupled to receive, analyze, input, modify, create and send data to and from the system 1210, including to and from one or more enterprise applications 1238 running on the system 1210. In some embodiments, at least one software application 1238 running on one or more processors 1232 can be configured to be coupled for communication over networks 1239a, 1239b through the internet 1239b. In some embodiments, one or more wired or wirelessly coupled components of the network 1239a, 1239b can include one or more resources for data storage. For example, in some embodiments, this can include any other form of computer readable media in addition to the computer readable media 1236 for storing information, and can include any form of computer readable media for communicating information from one electronic device to another electronic device.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Modifications to the illustrated embodiments and the generic principles herein can be applied to all embodiments and applications without departing from embodiments of the system. Also, it is understood that features from some embodiments presented herein are combinable with other features according to some embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Furthermore, acting as Applicant's own lexicographer, Applicant imparts the additional meaning to the following terms:

“Substantially,” “approximately,” and “about” when used in conjunction with a value and/or measurement encompasses a difference of 5% or less of the same unit and/or scale of the element for which it is associated. In some embodiments, “substantially,” “approximately,” and “about” are defined as presented in the specification in accordance with some embodiments.

“Simultaneously” includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

The use of and/or, in terms of “A and/or B,” means one option could be “A and B” and another option could be “A or B.” Such an interpretation is consistent with the USPTO Patent Trial and Appeals Board ruling in ex parte Gross, where the Board established that “and/or” means element A alone, element B alone, or elements A and B together.

As used herein, some embodiments recited with term “can” or “may” or derivations there of (e.g., the system display can show X) is for descriptive purposes only and “can” or “may” is understood to be interchangeable with “configured to” (e.g., the system display is configured to show X) for defining the metes and bounds of the system

The previous detailed description is to be read with reference to the figures, in which like elements in different figures have like representations. The figures, which are not necessarily to scale, depict some embodiments and are not intended to limit the scope of embodiments of the system.

Any of the operations described herein that form part of the invention are useful machine operations. The system also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

The embodiments of the system can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A system for collecting data from multinational clinical trials comprising:
one or more computers comprising one or more processors and one or more computer readable media, the one or more computer readable media comprising processor executable instructions that when executed by the one or more processors implement:
a graphical user interface (GUI) configured to display a form, the form configured to display and/or collect clinical trial information, and
a translation module;
wherein the clinical trial information comprises an input language and a translated language; and
wherein the system is configured to receive the input language from the form and translate the input language to the translated language.

2. The system of claim 1,
wherein the one or more computer readable media further comprise processor executable instructions that when executed by the one or more processors implement:
a geolocation module,
a comparison module, and
a language list;
wherein the language list comprises a list of one or more languages used in one or more geographical locations;
wherein the geolocation module is configured to receive a geolocation of one or more remote computers displaying the form;
wherein the comparison module is configured to compare the geolocation to the one or more geographical locations; and
wherein if the geolocation matches the one or more geographical locations, the system is configured to display at least a portion of the form in at least one of the one or more languages.

3. The system of claim 2,
wherein the at least a portion of the form is a data collection form; and
wherein the data collection form is configured to display text associated with the data collection form in at least one of the one or more languages if the geolocation matches the one or more geographical locations.

4. The system of claim 3,
wherein the system is configured to enable a user to translate the input language to the translated language.

5. The system of claim 4,
wherein the form is configured to enable a user to present text associated with the data collection form in multiple languages.

6. The system of claim 5,
wherein at least one of the multiple languages is the translated language.

7. The system of claim 3,
wherein the data collection form is configured to display text associated with the data collection form in the input language in response to the input language being detected and/or identified.

8. The system of claim 3,
wherein at least one of the multiple languages is the translated language; and
wherein at least one of the one or more languages is the input language.

9. A system for collecting data from multinational clinical trials comprising:
one or more computers comprising one or more processors and one or more computer readable media, the one or more computer readable media comprising processor executable instructions that when executed by the one or more processors implement:
a geolocation module,
a comparison module,
a language list, and
a form;
wherein the form is configured to display and/or collect clinical trial information;
wherein the language list comprises a list of one or more languages used in one or more geographical locations;
wherein the geolocation module is configured to receive a geolocation of one or more remote computers displaying the form;
wherein the comparison module is configured to compare the geolocation to the one or more geographical locations; and
wherein if the geolocation matches the one or more geographical locations, the system is configured to display at least a portion of the form in at least one of the one or more languages.

10. The system of claim 9,
wherein the one or more computer readable media further comprise processor executable instructions that when executed by the one or more processors implement:
a graphical user interface (GUI), and
a translation module;
wherein the GUI is configured to display the form and/or receive input for the form;
wherein the clinical trial information comprises an input language and a translated language; and
wherein the form configured to receive the input language and translate the input language to the translated language.

11. The system of claim 10,
wherein the form is configured to enable a user to present text associated with the data collection form in multiple languages.

12. The system of claim 11,
wherein at least one of the multiple languages is the translated language.

13. The system of claim 11,
wherein at least one of the one or more languages is the input language.

14. The system of claim 11,
wherein at least one of the multiple languages is the translated language; and
wherein at least one of the one or more languages is the input language.

15. The system of claim 9,
wherein the at least a portion of the form is a data collection form; and
wherein the data collection form is configured to display data entered into the data collection form in at least two of the one or more languages if the geolocation matches the one or more geographical locations.

16. The system of claim 15,
wherein the at least two of the one or more languages are each a translation of the input language; and
wherein the input language comprises is a language selected by a user.

17. The system of claim 15,
wherein the one or more computer readable media further comprise processor executable instructions that when executed by the one or more processors implement:
a graphical user interface (GUI), and
a translation module;
wherein the GUI is configured to display the form and/or receive input for the form;
wherein the clinical trial information comprises an input language and a translated language; and
wherein the form configured to receive the input language and translate the input language to the translated language.

18. The system of claim 17,
wherein the system is configured to enable a user to translate the input language to the translated language.
